(19) United States
(12) Patent Application Publication
(10) Pub. No.: US 2014/0288634 A1
(43) Pub. Date: Sep. 25, 2014

(54) DOUBLE-LAYER STENT

(71) Applicant: Alon Shalev, Ra'anana (IL)

(72) Inventor: Alon Shalev, Ra'anana (IL)

(73) Assignee: ENDOSPAN LTD., Herzilyia Pituach (IL)

(21) Appl. No.: 14/241,793

(22) PCT Filed: Aug. 12, 2012
(86) PCT No.: PCT/IL2012/000300

§ 371 Date: Apr. 23, 2014

Related U.S. Application Data
(60) Provisional application No. 61/529,931, filed on Sep. 1, 2011.

(51) Int. Cl. A61F 2/852; A61F 2/848; A61F 2/90

(52) U.S. Cl. CPC A61F 2/852; A61F 2/90; A61F 2/848; A61F 2002/826
USPC 623/1.16

(57) ABSTRACT

A multi-component endovascular stent system (10) includes an outer generally tubular stent (20), configured to assume radially-compressed and radially-expanded states, and an inner generally tubular stent (22), including a braided metal wire mesh (24) including wire, and configured to assume a radially-compressed and longitudinally-elongated state and a radially-expanded and longitudinally-contracted state. The inner stent (22) is configured to be nested in the outer stent (20), such that the inner and the outer stents (22, 20) are fixed together when the inner stent (22) is in its radially-expanded and longitudinally-contracted state and the outer stent (20) is in its radially-expanded state.

### CROSS-REFERENCE TO RELATED APPLICATIONS

The present application claims priority from U.S. Provisional Application 61/529,931, filed Sep. 1, 2011, which is assigned to the assignee of the present application and is incorporated herein by reference.

### FIELD OF THE APPLICATION

The present application relates generally to prostheses and surgical methods, and specifically to tubular prostheses, including endovascular grafts and stent-grafts, and surgical techniques for using the prostheses to maintain patency of body passages such as blood vessels, and treating aneurysms.

### BACKGROUND OF THE APPLICATION

Endovascular prostheses are sometimes used to treat aortic aneurysms. Such treatment includes implanting a stent or stent-graft within the diseased vessel to bypass the anomaly. An aneurysm is a sac formed by the dilation of the wall of the artery. Aneurysms may be congenital, but are usually caused by disease or, occasionally, by trauma. Aortic aneurysms which commonly form between the renal arteries and the iliac arteries are referred to as abdominal aortic aneurysms (“AAAs”). Other aneurysms occur in the aorta, such as thoracic aortic aneurysms (“TAAs”) and aortic uni-iliac (“AUI”) aneurysms. A TAA may occur downstream the aortic arch, i.e., in the descending aorta. Alternatively, a TAA may occur in the aortic arch itself, where the aorta branches to supply the brachiocephalic, left carotid and subclavian arteries, or may occur in the ascending aorta.

Endo-Vascular Aneurysm Repair (EVAR) has transformed the practice of treatment of aortic aneurysms from an open surgical approach to a much less invasive surgical approach. The first step of an endovascular intervention usually requires introducing a delivery system into the vasculature of a subject. If the crossing profile, i.e., the external diameter, of the delivery system is 14 Fr or lower (3 Fr=1 millimeter), a true percutaneous approach may be used, because vascular closure devices are available for proper closure of such puncture sites. If the crossing profile at least 15-16 Fr, a vascular cut-down is usually required in advance as a preparatory step to introduction of the delivery system.

Endovascular systems for treatment of supra-renal aneurysms generally require the preparatory step of a vascular cut-down. A cut-down is the localized surgical exposure of blood vessels for accessing the subject's vasculature. For example, most surgical cut-downs used in EVAR procedures are performed in the vicinity of the pubis, exposing the iliac arteries. Surgical cut-downs have related complications and co-morbidities, including difficulty in controlling bleeding at the access site, false aneurysms, and vascular obstruction. It is therefore desirable to use a purely percutaneous approach, instead of a vascular cut-down.

Endovascular stent-grafts for treating the thoracic aorta usually require a 20-22 Fr delivery system, because of the large amount of graft material indicated by the diameter of the aorta above the level of the renal arteries (30-40 mm diameter or more in some subjects). Currently used graft materials are PET (Poly Ethylene Therephtalate) and ePTFE (expanded Poly-Tetra-Fluoro-Ethylene). The thickness and circumferential length of the graft have the most substantial effect on the crossing profile of an endovascular system. The use of thinner graft materials generally reduces long-term durability of the graft material.

### SUMMARY OF APPLICATIONS

In some applications of the present invention, a multi-component endovascular stent system comprises an outer generally tubular stent, and an inner generally tubular stent. The outer and inner stents are configured to be assembled in situ by nesting the inner stent in the outer stent. In general, the outer stent provides anchoring (fixation and migration resistance) for the stent system within an aneurysmal body lumen, such as an aneurysmal blood vessel, while the inner stent provides a generally blood-impervious fluid flow path through the stent system.

Graft material generally has the most significant effect on the crossing profile of a stent-graft. Because each of the outer and inner stents comprises substantially less graft material (typically no graft material) than a typical single-component full-circumference stent-graft, the stents typically have relatively small crossing profiles. In addition, the inner stent is highly elongated and radially-compressed during endoluminal delivery, thereby substantially reducing its crossing profile. The outer and inner stents thus can typically be deployed using catheters having a diameter of no more than 14 Fr. This generally enables the use of a true percutaneous surgical technique, without the need for a vascular cut-down.

The inner stent comprises a braided wire mesh, which typically comprises a metal, such as a super-elastic alloy and/or a shape-memory alloy, e.g., Nitinol. The inner stent is configured to assume (a) a radially-compressed and longitudinally-elongated state, for low-crossing-profile endoluminal delivery, and (b) a radially-expanded and longitudinally-contracted state, for secure fixation within the outer stent. The inner stent has an elongated length when in its radially-compressed and longitudinally-elongated state, and a contracted length when in its radially-expanded and longitudinally-contracted state. Typically, the inner stent is configured such that a ratio of the elongated length to the contracted length is at least 4, such as at least 8, at least 10, or at least 12.

The inner stent is configured to be substantially blood-impervious when in its radially-expanded and longitudinally-contracted state within the outer stent, beginning no later than four weeks after implantation of the stent system in a body lumen, such as a blood vessel. Typically, the inner stent does not comprise a graft member, i.e., the multi-component endovascular stent system does not comprise any graft material attached to the inner stent at least when the inner stent is not nested in the outer stent. The braided wire mesh of the inner stent is sufficiently dense to provide blood-impermeability, thereby obviating the need for a graft member.

For some applications, in order to increase the blood-imperviousness of the inner stent and/or the rate of haemostatic sealing, the inner stent further comprises a thrombogenic agent that coats the wire of the braided wire mesh. The thrombogenic agent facilitates quicker haemostatic sealing of the inner stent after implantation.

For some applications, the stent system is deployed in the aorta or another blood vessel for treating an aneurysm. During an implantation procedure, the outer stent is introduced into the aorta, while restrained in the stent's radially-compressed state in a delivery catheter. The outer stent is deployed in the aorta by transitioning the outer stent to its radially-expanded state.

Similarly, the inner stent is initially positioned in a delivery catheter, restrained in the stent's radially-compressed and longitudinally-elongated state by the catheter. The inner stent, while positioned in the delivery catheter, is transvascularly introduced into the aorta. The inner stent, while in the delivery catheter, is introduced at least partially into the outer stent. The delivery catheter is proximally withdrawn, releasing the inner stent in the aorta, within the outer stent, and transitioning the inner stent to the radially-expanded and longitudinally-contracted state thereof. Upon being fully deployed, the inner stent is at least partially (e.g., entirely) nested within the outer stent, such that the inner and the outer stents are fixed together.

There is therefore provided, in accordance with an application of the present invention, apparatus including a multi-component endovascular stent system, which includes:

an outer generally tubular stent, which is configured to assume radially-compressed and radially-expanded states; and

an inner generally tubular stent, which includes a braided metal wire mesh including wire, and which is configured to assume a radially-compressed and longitudinally-elongated state and a radially-expanded and longitudinally-contracted state,

wherein the inner stent is configured to be nested in the outer stent, such that the inner and the outer stents are fixed together when the inner stent is in its radially-expanded and longitudinally-contracted state and the outer stent is in its radially-expanded state, and

wherein the inner stent (a) has (i) an elongated length when in its radially-compressed and longitudinally-elongated state and (ii) a contracted length when in its radially-expanded and longitudinally-contracted state within the outer stent when the outer stent is unconstrained in its radially-expanded state, and (b) is configured such that a ratio of the elongated length to the contracted length is at least 4.

For some applications, the ratio is at least 8, such as at least 10, e.g., at least 12.

For some applications, the inner stent includes a super-elastic alloy. Alternatively or additionally, for some applications, the inner stent includes a shape-memory alloy. Alternatively or additionally, for some applications, the inner stent includes Nitinol.

For some applications, the inner stent further includes a thrombogenic agent that coats the wire of the braided wire mesh. For some applications, the thrombogenic agent is selected from the group including: gelatin, collagen, von Willebrand factor, thrombospondin, tissue factor, a phospholipid, platelet activating factor, an analogue of platelet activating factor, fibrin, factor V, factor IX, an antiphospholipid antibody, a portion of an antiphospholipid antibody, copper, an alloy of copper, platinum, and an alloy of platinum.

For some of the applications described above, the multi-component endovascular stent system does not comprise any graft material attached to the inner stent at least when the inner stent is not nested in the outer stent.

For any of the applications described above, the inner stent may have an expanded diameter when in its radially-expanded and longitudinally-contracted state within the outer stent when the outer stent is unconstrained in its radially-expanded state, the inner stent may have a contracted diameter when in its radially-compressed and longitudinally-elongated state, and a ratio of the expanded diameter to the contracted diameter may be at least 1.1, such as at least 1.5.

For any of the applications described above, the contracted length of the inner stent may be equal to between 90% and 110% of a length of the outer stent when unconstrained in its radially-expanded state.

For any of the applications described above, a wall thickness of the inner stent when in its radially-expanded and longitudinally-contracted state within the outer stent, when unconstrained in its radially-expanded state, may be no more than 200% of a wall thickness of the outer stent when unconstrained in its radially-expanded state.

For any of the applications described above, an expanded surface coverage ratio of the inner stent when in its radially-expanded and longitudinally-contracted state within the outer stent, when the outer stent is unconstrained in its radially-expanded state, may be equal to at least 25% of a contracted surface coverage ratio of the inner stent when in its radially-compressed and longitudinally-elongated state. For example, the expanded surface coverage ratio may be equal to at least 50% of the contracted surface coverage ratio, e.g., at least 100% of the contracted surface coverage ratio.

For any of the applications described above, an expanded surface coverage ratio of the inner stent when in its radially-expanded and longitudinally-contracted state within the outer stent, when the outer stent is unconstrained in its radially-expanded state, may be equal to at least 30% of an expanded surface coverage ratio of the outer stent when unconstrained in its radially-expanded state.

For any of the applications described above, an expanded surface coverage ratio of the inner stent when in its radially-expanded and longitudinally-contracted state within the outer stent, when the outer stent is unconstrained in its radially-expanded state, may be equal to at least 20%, such as at least 50%.

For some applications, the braided wire mesh of the inner stent, if projected onto a plane when the inner stent is in its radially-expanded and longitudinally-contracted state within the outer stent, when the outer stent is unconstrained in its radially-expanded state, would define a plurality of openings, and, for each of at least 90% of the openings, every point on the plane within the projected opening is within 0.2 mm of a border of the opening on the plane, which border is defined by the braided wire mesh projected onto the plane.

For some applications, the braided wire mesh of the inner stent, if projected onto a plane when the inner stent is in its radially-expanded and longitudinally-contracted state within the outer stent, when the outer stent is unconstrained in its radially-expanded state, would define a plurality of openings, which have a median size of at least 100 square micrometers.

For any of the applications described above, the inner stent may be configured to be substantially blood-impermeable when in its radially-expanded and longitudinally-contracted state within the outer stent, beginning no later than four weeks after implantation of the outer and the inner stents in a body lumen.

For any of the applications described above, the outer stent may be shaped so as to define a plurality of outwardly-protruding fixation members, which are configured to facilitate fixation of the outer stent to an inner wall of a body lumen.

For any of the applications described above, the outer stent may be shaped so as to define a plurality of inwardly-protruding fixation members, which are configured to facilitate coupling of the inner stent to the outer stent. For some applications, at least one of the inwardly-protruding fixation members includes a barb. For some applications, the plurality of inwardly-protruding fixation members includes at least a plurality of proximally-disposed fixation members, disposed near a proximal end of the outer stent, and a plurality of distally-disposed fixation members, disposed near a distal end of the outer stent. For some applications, the proximally-disposed fixation members include respective distally- and inwardly-oriented barbs. For some applications, the distally-disposed fixation members include respective proximally- and inwardly-oriented barbs.

For any of the applications described above, the wire of the braided wire mesh may have a diameter of between 50 and 200 micrometers.

For any of the applications described above, each of the outer stent and the inner stent may further include one or more respective radiopaque markers.

For any of the applications described above, the outer stent may include a plurality of structural stent elements, which are arranged as a plurality of circumferential bands. For some applications, the structural stent elements are interconnected. For some applications, the outer stent further includes a polymeric fabric, which connects the circumferential bands. For some applications, the polymeric fabric covers less than an entire circumference of the outer stent along at least a portion of the outer stent. For some applications, the polymeric fabric circumscribes a circumferential arc that is substantially constant along at least the portion. Alternatively, for some applications, the polymeric fabric circumscribes a circumferential arc that has an arc angular center that varies along at least the portion, thereby providing the polymeric fabric with a generally helical shape.

There is further provided, in accordance with an application of the present invention, a method for treating an aneurysm in a body lumen of a human subject, the method including:

transvascularly introducing an outer generally tubular stent into the lumen, while the outer stent is in a radially-compressed state thereof;

deploying the outer stent in the lumen longitudinally spanning the aneurysm by transitioning the outer stent to a radially-expanded state thereof;

transvascularly introducing, into the lumen and at least partially into the outer stent, an inner generally tubular stent that includes a braided metal wire mesh including wire, while the inner stent in a radially-compressed and longitudinally-elongated state thereof, in which the inner stent has an elongated length; and

deploying the inner stent in the lumen at least partially nested within the outer stent such that the inner and the outer stents are fixed together, by transitioning the inner stent to a radially-expanded and longitudinally-contracted state thereof, in which the inner stent has a contracted length that is no more than 25% of the elongated length.

For some applications, the contracted length is no more than 12.5% of the elongated length, such as no more than 10% of the elongated length, e.g., no more than 8.3% of the elongated length.

For some applications, transvascularly introducing the inner stent includes transvascularly introducing the inner stent while the inner stent is constrained in its radially-compressed and longitudinally-elongated state in a delivery catheter, and deploying the inner stent includes deploying the inner stent from the delivery catheter.

For some applications, the inner stent includes a super-elastic alloy.

Alternatively or additionally, for some applications, the inner stent includes a shape-memory alloy. Alternatively or additionally, for some applications, the inner stent includes Nitinol.

For some applications, the inner stent further includes a thrombogenic agent that coats the wire of the braided wire mesh. For some applications, the thrombogenic agent is selected from the group including: gelatin, collagen, von Willebrand factor, thrombospondin, tissue factor, a phospholipid, platelet activating factor, an analogue of platelet activating factor, fibrin, factor V, factor IX, an antiphospholipid antibody, a portion of an antiphospholipid antibody, copper, an alloy of copper, platinum, and an alloy of platinum.

For some applications, transvascularly introducing the inner stent comprises transvascularly introducing the inner stent with no graft material attached to the inner stent.

For some applications, transvascularly introducing the inner stent includes transvascularly introducing the inner stent in its radially-compressed and longitudinally-elongated state, in which the inner stent has a contracted diameter, and deploying the inner stent includes transitioning the inner stent to its radially-expanded and longitudinally-contracted state within the outer stent when in its radially-expanded state, in which state the inner stent has an expanded diameter, which is at least 10% greater than the contracted diameter, such as at least 50% greater than the contracted diameter.

For some applications, the contracted length of the inner stent when deployed is equal to between 90% and 110% of a length of the outer stent deployed in its radially-expanded state.

For some applications, deploying the inner stent includes transitioning the inner stent its radially-expanded and longitudinally-contracted state within the outer stent when deployed in its radially-expanded state, in which a wall thickness of the inner stent, is no more than 200% of a wall thickness of the outer stent when deployed in its radially-expanded state.

For some applications, transvascularly introducing the inner stent includes transvascularly introducing the inner stent in its radially-compressed and longitudinally-elongated state, in which the inner stent has a contracted surface coverage ratio; and deploying the inner stent includes transitioning the inner stent to its radially-expanded and longitudinally-contracted state within the outer stent when in its radially-expanded state, in which state the inner stent has an expanded surface coverage ratio, which is equal to at least 25% of the contracted surface coverage ratio.

For example, the expanded surface coverage ratio may be equal to at least 50% of the contracted surface coverage ratio, such as at least 100% of the contracted surface coverage ratio.

For some applications, deploying the outer stent includes transitioning the outer stent to its radially-expanded state, in which the outer stent has an expanded surface coverage ratio; and deploying the inner stent includes transitioning the inner stent to its radially-expanded and longitudinally-contracted state within the outer stent when in its radially-expanded state, in which state the inner stent has an expanded surface coverage ratio, which is equal to at least 30% of the expanded surface coverage ratio of the outer stent.

For some applications, deploying the inner stent includes transitioning the inner stent to its radially-expanded and longitudinally-contracted state within the outer stent when in its radially-expanded state, in which state the inner stent has an expanded density, which is equal to at least 20%, such as at least 50%.

For some applications, deploying the inner stent comprises transitioning the inner stent to its radially-expanded and longitudinally-contracted state within the outer stent when in its radially-expanded state, in which state the braided wire mesh of the inner stent, if projected onto a plane, would define a plurality of openings, and, for each of at least 90% of the openings, every point on the plane within the projected opening is within 0.2 mm of a border of the opening on the plane, which border is defined by the braided wire mesh projected onto the plane.

For some applications, deploying the inner stent comprises transitioning the inner stent to its radially-expanded and longitudinally-contracted state within the outer stent when in its radially-expanded state, in which state the braided wire mesh of the inner stent, if projected onto a plane, would define a plurality of openings, which have a median size of at least 100 square micrometers.

For some applications, deploying the inner stent includes transitioning the inner stent to its radially-expanded and longitudinally-contracted state within the outer stent when in its radially-expanded state, in which state the inner stent substantially blood-impermeable, beginning no later than four weeks after implantation of the outer and the inner stents in the body lumen.

For some applications, the outer stent is shaped so as to define a plurality of outwardly-protruding fixation members, and deploying the outer stent includes using the fixation members to facilitate fixation of the outer stent to an inner wall of the body lumen.

For some applications, the outer stent is shaped so as to define a plurality of inwardly-protruding fixation members, and deploying the inner stent includes using the fixation members to facilitate coupling of the inner stent to the outer stent. For some applications, at least one of the inwardly-protruding fixation members includes a barb.

For some applications, the wire of the braided wire mesh has a diameter of between 50 and 200 micrometers.

For some applications, each of the outer stent and the inner stent further includes one or more respective radiopaque markers.

For some applications, the body lumen is a blood vessel, and transvascularly introducing the outer and the inner stents includes transvascularly introducing the outer and the inner stents in the blood vessel. For example, the blood vessel may be an aorta, and transvascularly introducing the outer and the inner stents may include transvascularly introducing the outer and the inner stents in the aorta. For some applications, the method further includes identifying the subject as suffering from an aortic aneurysm, and transvascularly introducing and deploying the outer and the inner stents includes transvascularly introducing and deploying the outer and the inner stents responsively to the identifying.

The present invention will be more fully understood from the following detailed description of embodiments thereof, taken together with the drawings, in which:

### BRIEF DESCRIPTION OF THE DRAWINGS

FIGS. 1A-B are schematic illustrations of an outer stent of a multi-component endovascular stent system, in accordance with an application of the present invention;

FIGS. 2A-B are schematic illustrations of an inner stent of the multi-component endovascular stent system of FIGS. 1A-B, in accordance with an application of the present invention;

FIG. 3 is a schematic illustration of the multi-component endovascular stent system of FIGS. 1A-B and 2A-B in an assembled state, in accordance with an application of the present invention;

FIGS. 4A-E are schematic illustrations of an exemplary method of deploying the multi-component endovascular stent system of FIGS. 1A-B and 2A-B, in accordance with an application of the present invention; and

FIG. 5 is a schematic illustration of another configuration of the multi-component endovascular stent system of FIGS. 1A-B and 2A-B, in accordance with an application of the present invention.

### DETAILED DESCRIPTION OF APPLICATIONS

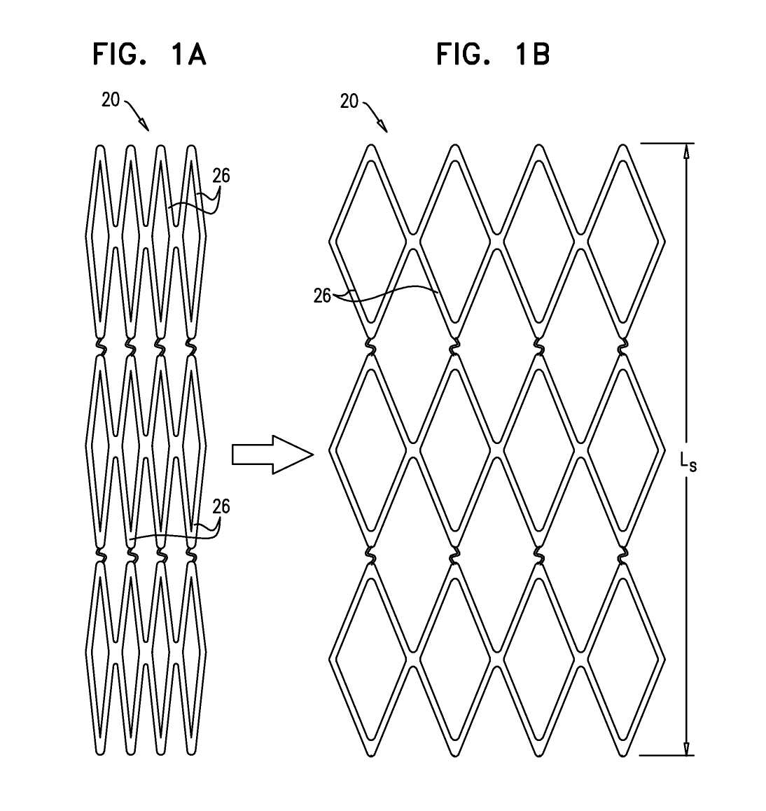

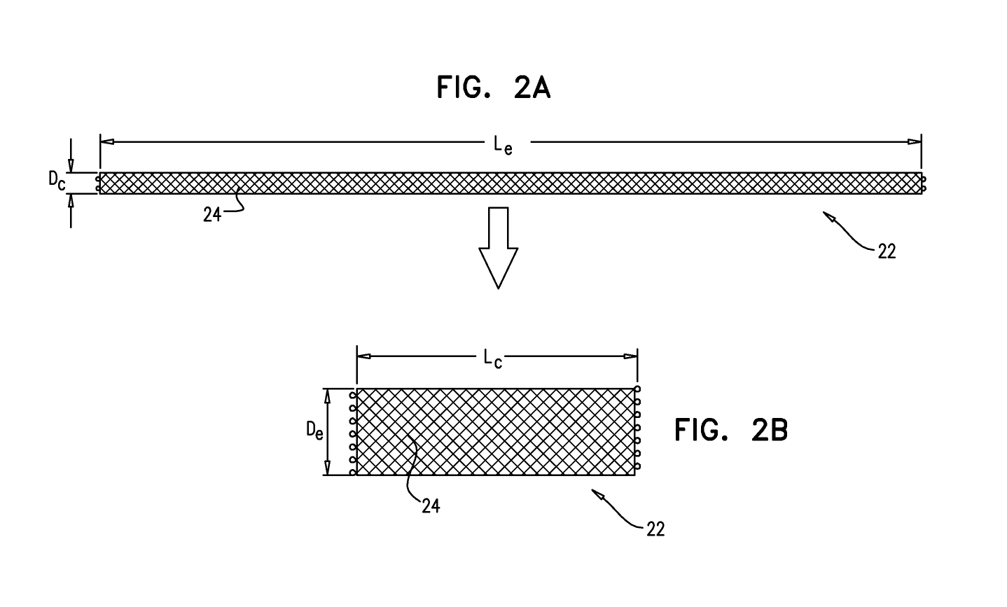

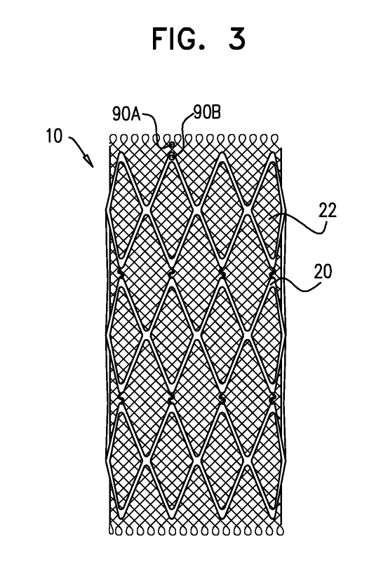

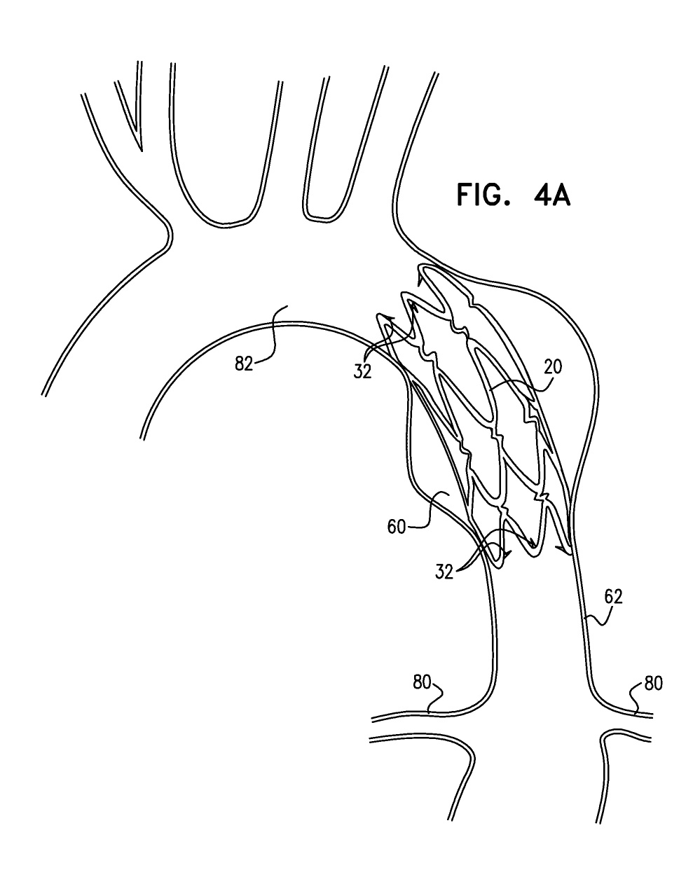

FIGS. 1A-B, 2A-B, and 3 are schematic illustrations of a multi-component endovascular stent system 10, in accordance with an application of the present invention. FIGS. 1A-B and 2A-B show disassembled components of the stent system, and FIG. 3 shows the stent system assembled. Stent system 10 comprises an outer generally tubular stent 20, shown alone in FIGS. 1A-B, and an inner generally tubular stent 22, shown alone in FIGS. 2A-B (it is noted that FIGS. 1A-B and 2A-B are not drawn to the same scale). Stent system 10 is assembled in situ by nesting inner stent 22 in outer stent 20, as shown in FIG. 3. In general, outer stent 20 provides anchoring (fixation and migration resistance) for stent system 10 within an aneurysmal body lumen, such as an aneurysmal blood vessel, while inner stent 22 provides a generally blood-impervious fluid flow path through stent system 10. Outer stent 20 is sufficiently rigid to provide strength for stent system 10.

Outer stent 20 is configured to assume (a) a radially-compressed state, as shown in FIG. 1A, for low-crossing-profile endoluminal delivery, and (b) a radially-expanded state, as shown in FIG. 1B, for secure fixation in a body lumen, such as a blood vessel. Typically, outer stent 20 comprises a metal, such as a super-elastic alloy and/or a shape-memory alloy, e.g., Nitinol. Typically, outer stent 20 is self-expanding. For some applications, outer stent 20 is heat-set to assume the radially-expanded state.

Outer stent 20 typically comprises a plurality of structural stent elements (struts) 26, which, for some applications, are arranged as a plurality of circumferential bands. For some applications, at least some of (e.g., all of) structural stent elements 26 are interconnected, while for other applications, at least a portion of (e.g., all of) the structural stent elements are not interconnected. For example, outer stent 20 may further comprise a polymeric fabric, which connects the circumferential bands. For some applications, the polymeric fabric covers less than an entire circumference of outer stent 20 along at least a portion of the outer stent (e.g., at least a portion that includes all of the structural stent elements), or along the entire length of the outer stent. For example, the polymeric fabric may have an elongated rectangular shape, which circumscribes a circumferential arc along a covered-length portion of outer stent 20. For some applications, the polymeric fabric circumscribes a circumferential arc that is substantially constant along at least a portion of the outer stent. Alternatively, the circumferential arc has an angular center that varies along at least a portion of the outer stent, thereby providing the polymeric fabric with a generally helical shape. Alternatively, outer stent 20 does not comprise a graft member.

As shown in FIGS. 2A-B, inner stent 22 comprises a braided wire mesh 24, which typically comprises a metal, such as a super-elastic alloy and/or a shape-memory alloy, e.g., Nitinol. Inner stent 22 is configured to assume (a) a radially-compressed and longitudinally-elongated state, as shown in FIG. 2A, for low-crossing-profile endoluminal delivery, and (b) a radially-expanded and longitudinally-contracted state, as shown in FIG. 2B, for secure fixation within outer stent 20. Typically, inner stent 22 is self-expanding. For some applications, inner stent 22 is heat-set to assume the radially-compressed and longitudinally-elongated state. For some applications, the wire of wire mesh 24 has a diameter of at least 50 micrometers, no more than 200 micrometers, and/or between 50 and 200 micrometers.

The blood-permeability of inner stent 22 decreases as the diameter of inner stent increases when the inner stent transitions from its radially-contracted and longitudinally-elongated state (FIG. 2A) to its radially-expanded and longitudinally-contracted state (FIG. 2B). Inner stent 22 is configured to be substantially blood-impervious when in its radially-expanded and longitudinally-contracted state within outer stent 20, beginning no later than four weeks after implantation of the outer and the inner stents in a body lumen, such as a blood vessel. The inner stent is typically somewhat blood-permeable initially upon implantation. Over the first several days or weeks after implantation, the inner stent gradually becomes substantially blood-impervious as the small openings of braided wire mesh 24 become covered with proteins, cellular components, and platelets.

For some applications, in order to increase the blood-imperviousness of inner stent 22 and/or the rate of haemostatic sealing, the inner stent further comprises a thrombogenic agent that coats the wire of wire mesh 24 of the inner stent. The thrombogenic agent facilitates quicker haemostatic sealing of inner stent 22 after implantation. For some applications, the thrombogenic agent is selected from the group comprising: gelatin, collagen, von Willebrand factor, thrombospondin, tissue factor, a phospholipid, platelet activating factor, an analogue of platelet activating factor, fibrin, factor V, factor IX, an antiphospholipid antibody, a portion of an antiphospholipid antibody, copper, an alloy of copper, platinum, and an alloy of platinum.

Typically, inner stent 22 does not comprise a graft member, i.e., multi-component endovascular stent system 10 does not comprise any graft material attached to inner stent 22 at least when inner stent 22 is not nested in outer stent 20. Braided wire mesh 24 of the inner stent is sufficiently dense to provide blood-impermeability, thereby obviating the need for a graft member. For some applications, neither inner stent 22 nor outer stent 20 comprises a graft member.

As shown in FIG. 3, inner stent 22 is configured to be nested in outer stent 20, such that inner and outer stents 22 and 20 are fixed together when inner stent 22 is in its radially-expanded and longitudinally-contracted state and outer stent 20 is in its radially-expanded state. FIG. 3 shows outer stent 20 unconstrained in its radially-expanded state, and inner stent 22 in its radially-expanded and longitudinally-contracted state within outer stent 20.

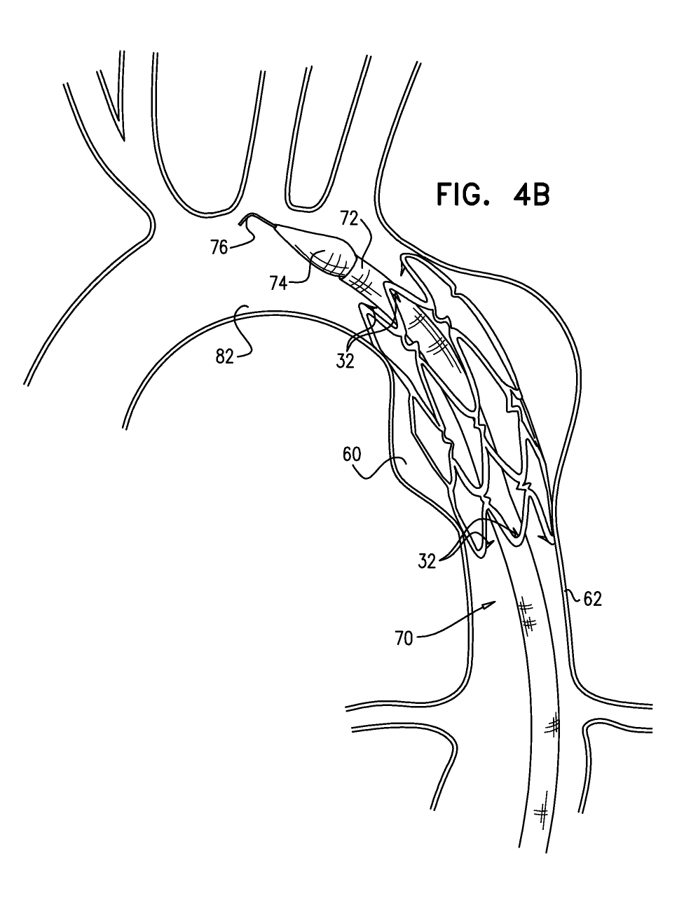

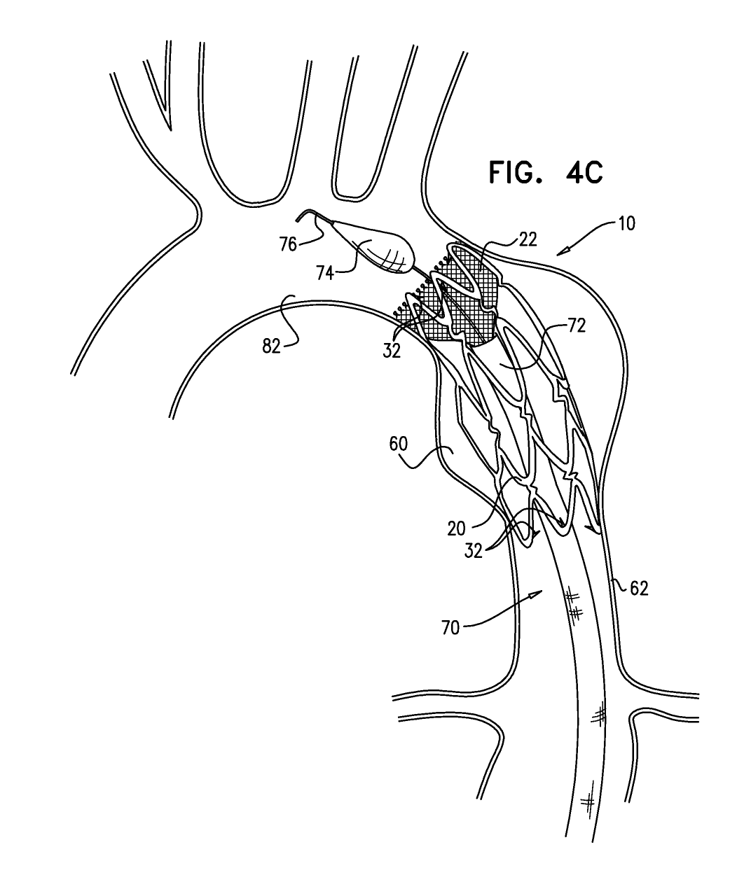

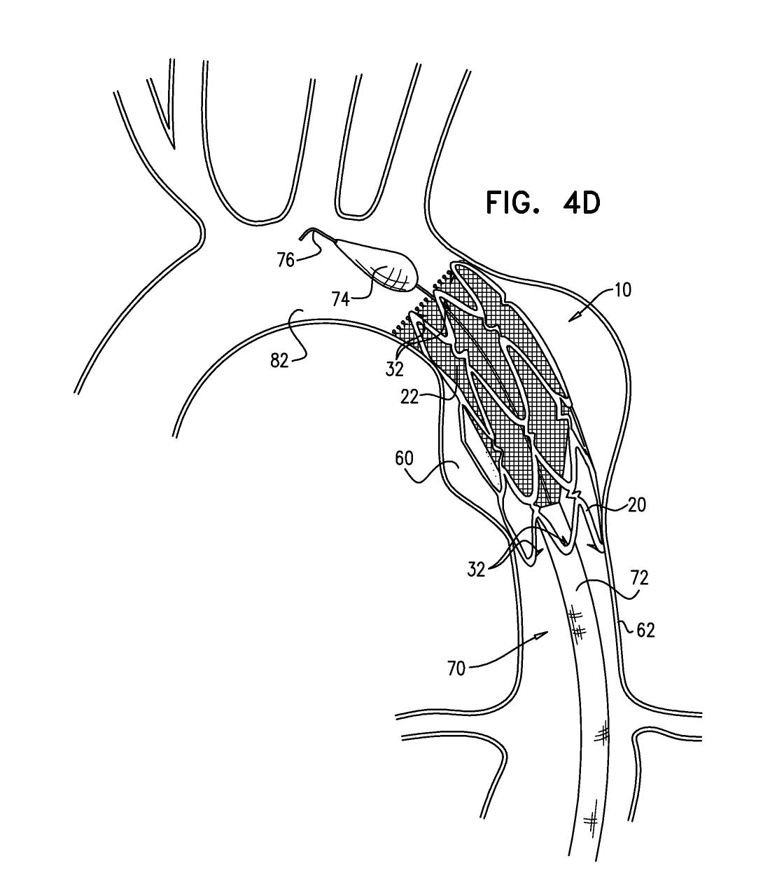

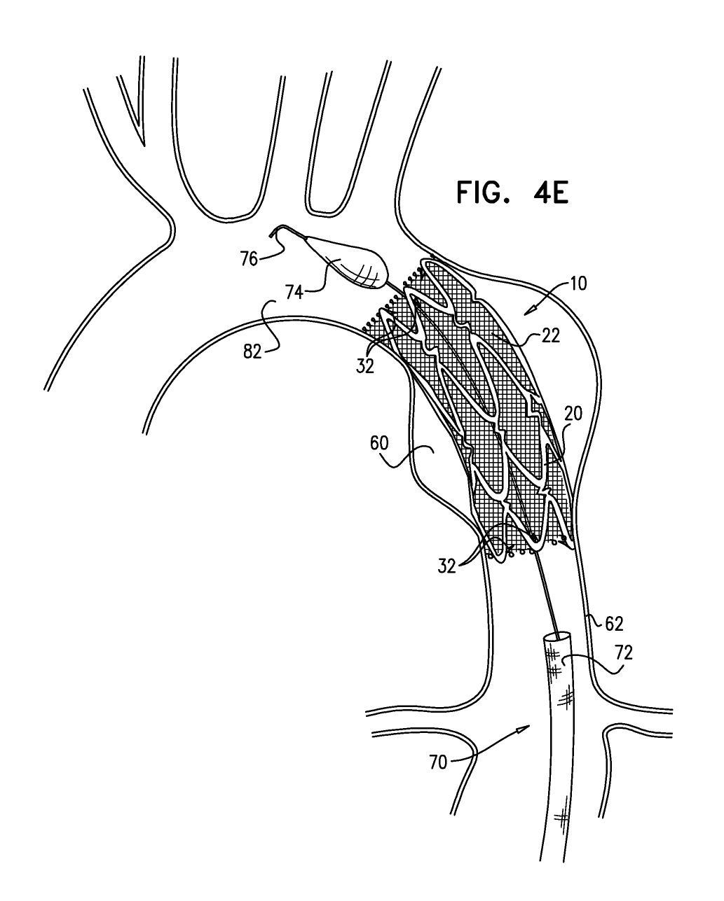

Inner stent 22 has (a) an elongated length Lₑ when in its radially-compressed and longitudinally-elongated state, as shown in FIG. 2A, and (b) a contracted length L_{c} when in its radially-expanded and longitudinally-contracted state. Typically, inner stent 22 is configured such that a ratio of elongated length Lₑ to contracted length L_{c} (i.e., a ratio of elongation) is at least 4, such as at least 6, at least 8, at least 10, or at least 12. For some applications, inner stent 22 has contracted length L_{c} when the inner stent is within outer stent 20 when the outer stent is unconstrained in its radially-expanded state, i.e., when no forces are applied to the outer stent by a delivery tool, a wall of a body lumen (e.g., a blood vessel), or otherwise, as shown in FIG. 3. Alternatively, inner stent 22 has contracted length L_{c} when within outer stent 20 when the outer stent is constrained by a wall of a body lumen, e.g., a blood vessel, for example as shown in FIG. 4E. Further alternatively, inner stent 22 has contracted length L_{c} when the inner stent is unconstrained, i.e., when no forces are applied to the inner stent by outer stent 20, a delivery tool, wall of a body lumen (e.g., blood vessel), or otherwise, as shown in FIG. 2B.

For some applications, inner stent 22 has (a) an expanded diameter Dₑ when in its radially-expanded and longitudinally-contracted state, and (b) a contracted diameter D_{c} when in its radially-compressed and longitudinally-elongated state, as shown in FIG. 2A. Typically, inner stent 22 is configured such that a ratio of expanded diameter Dₑ to contracted diameter D_{c} (i.e., a ratio of radial expansion of inner stent 22) is at least 1.1, such as at least 1.5, at least 2, at least 4, or at least 8. For some applications, inner stent 22 has expanded diameter Dₑ when the inner stent is within outer stent 20 when the outer stent is unconstrained in its radially-expanded state, i.e., when no forces are applied to the outer stent by a delivery tool, a wall of a body lumen (e.g., a blood vessel), or otherwise, as shown in FIG. 3. Alternatively, inner stent 22 has expanded diameter Dₑ when within outer stent 20 when the outer stent is constrained by a wall of a body lumen, e.g., a blood vessel, for example as shown in FIG. 4E. Further alternatively, inner stent 22 has expanded diameter Dₑ when the inner stent is unconstrained, i.e., when no forces are applied to the stent by outer stent 20, a delivery tool, a wall of a body lumen (e.g., a blood vessel), or otherwise, as shown in FIG. 2B.

For some applications, a ratio of (a) the ratio of elongation of inner stent 22 mentioned above to (b) the ratio of radial expansion of inner stent 22 mentioned above is between 0.5 and 4, such as between 0.5 and 1, between 1 and 2, or between 2 and 4. For example, the inner stent may have a ratio of elongation of 8, a ratio of radial expansion of 4, and a ratio of these two ratios of 2.

Alternatively or additionally, for some applications, an expanded surface coverage ratio of inner stent 22, when in its radially-expanded and longitudinally-contracted state within outer stent 20, is at least 25%, such as at least 50%, e.g., at least 100%, of a contracted surface coverage ratio of inner stent 22 when in its radially-compressed and longitudinally-elongated state. For some applications, inner stent 22 has the expanded surface coverage ratio when the inner stent is within outer stent 20 when the outer stent is unconstrained in its radially-expanded state, i.e., when no forces are applied to the outer stent by a delivery tool, a wall of a body lumen (e.g., a blood vessel), or otherwise, as shown in FIG. 3. Alternatively, inner stent 22 has the expanded surface coverage ratio when within outer stent 20 when the outer stent is constrained by a wall of a body lumen, e.g., a blood vessel, for example as shown in FIG. 4E. Further alternatively, inner stent 22 has the expanded surface coverage ratio when in its radially-expanded and longitudinally-contracted state when the inner stent is unconstrained, i.e., when no forces are applied to the stent by outer stent 20, a delivery tool, a wall of a body lumen (e.g., a blood vessel), or otherwise, as shown in FIG. 2B.

As used in the present application, including in the claims, a “surface coverage ratio” means a surface that the projection of the non-void portion of the stent covers. (It is noted that in a braided stent the wires are not in the same plane throughout the stent.)

Alternatively or additionally, for some applications, an expanded surface coverage ratio of inner stent 22, when in its radially-expanded and longitudinally-contracted state within outer stent 20, is equal to at least 30% of an expanded surface coverage ratio of outer stent 20 when unconstrained in its radially-expanded state. Alternatively, outer stent 20 has its expanded surface coverage ratio when constrained by a wall of a body lumen, e.g., a blood vessel. For some applications, inner stent 22 has its expanded surface coverage ratio when the inner stent is within outer stent 20 when the outer stent is unconstrained in its radially-expanded state, i.e., when no forces are applied to the outer stent by a delivery tool, a wall of a body lumen (e.g., a blood vessel), or otherwise, as shown in FIG. 3. Alternatively, inner stent 22 has its expanded surface coverage ratio when within outer stent 20 when the outer stent is constrained by a wall of a body lumen, e.g., a blood vessel, for example as shown in FIG. 4E. Further alternatively, inner stent 22 has its expanded surface coverage ratio when in its radially-expanded and longitudinally-contracted state when the inner stent is unconstrained, i.e., when no forces are applied to the stent by outer stent 20, a delivery tool, a wall of a body lumen (e.g., a blood vessel), or otherwise, as shown in FIG. 2B.

Alternatively or additionally, for some applications, an expanded surface coverage ratio of inner stent 22, when in its radially-expanded and longitudinally-contracted state within outer stent 20, is at least 20%, such as at least 50%. For some applications, inner stent 22 has this expanded surface coverage ratio when the inner stent is within outer stent 20 when the outer stent is unconstrained in its radially-expanded state, i.e., when no forces are applied to the outer stent by a delivery tool, a wall of a body lumen (e.g., a blood vessel), or otherwise, as shown in FIG. 3. Alternatively, inner stent 22 has this expanded surface coverage ratio when within outer stent 20 when the outer stent is constrained by a wall of a body lumen, e.g., a blood vessel, for example as shown in FIG. 4E. Further alternatively, inner stent 22 has this expanded surface coverage ratio when in its radially-expanded and longitudinally-contracted state when the inner stent is unconstrained, i.e., when no forces are applied to the stent by outer stent 20, a delivery tool, a wall of a body lumen (e.g., a blood vessel), or otherwise, as shown in FIG. 2B.

Alternatively or additionally, for some applications, braided wire mesh 24 of the inner stent, if projected onto a plane, would define a plurality of openings, which have a median size of at least 100 square micrometers, no more than 4 square millimeters, and/or between 100 square micrometers and 4 square millimeters, when the inner stent is in its radially-expanded and longitudinally-contracted state. Alternatively or additionally, for some applications, braided wire mesh 24, if projected onto a plane when the inner stent is in its radially-expanded and longitudinally-contracted state, would define a plurality of openings, and, for each of at least 90% of the openings, every point on the plane within the projected opening is within 0.3 mm, such as within 0.2 mm, of a border of the opening on the plane, which border is defined by the braided wire mesh projected onto the plane. For some applications, the openings have this median size when the inner stent is within outer stent 20 when the outer stent is unconstrained in its radially-expanded state, i.e., when no forces are applied to the outer stent by a delivery tool, a wall of a body lumen (e.g., a blood vessel), or otherwise, as shown in FIG. 3. Alternatively, the openings have this median size when the inner stent is within outer stent 20 when the outer stent is constrained by a wall of a body lumen, e.g., a blood vessel, for example as shown in FIG. 4E. Further alternatively, the openings have this median size when the inner stent is in its radially-expanded and longitudinally-contracted state when the inner stent is unconstrained, i.e., when no forces are applied to the stent by outer stent 20, a delivery tool, a wall of a body lumen (e.g., a blood vessel), or otherwise, as shown in FIG. 2B.

Alternatively or additionally, for some applications, a wall thickness of inner stent 22 when in its radially-expanded and longitudinally-contracted state, is no more than 200% of a wall thickness of outer stent 20 when unconstrained in its radially-expanded state. For some applications, inner stent 22 has this wall thickness when the inner stent is within outer stent 20 when the outer stent is unconstrained in its radially-expanded state, i.e., when no forces are applied to the outer stent by a delivery tool, a wall of a body lumen (e.g., a blood vessel), or otherwise, as shown in FIG. 3. Alternatively, inner stent 22 has this wall thickness when within outer stent 20 when the outer stent is constrained by a wall of a body lumen, e.g., a blood vessel, for example as shown in FIG. 4E. Further alternatively, inner stent 22 has this wall thickness when in its radially-expanded and longitudinally-contracted state when the inner stent is unconstrained, i.e., when no forces are applied to the stent by outer stent 20, a delivery tool, a wall of a body lumen (e.g., a blood vessel), or otherwise, as shown in FIG. 2B.

Alternatively or additionally, for some applications, contracted length L_{c} of inner stent 22 is equal to between 90% and 110% of a length Lₛ of outer stent 20 when unconstrained in its radially-expanded state.

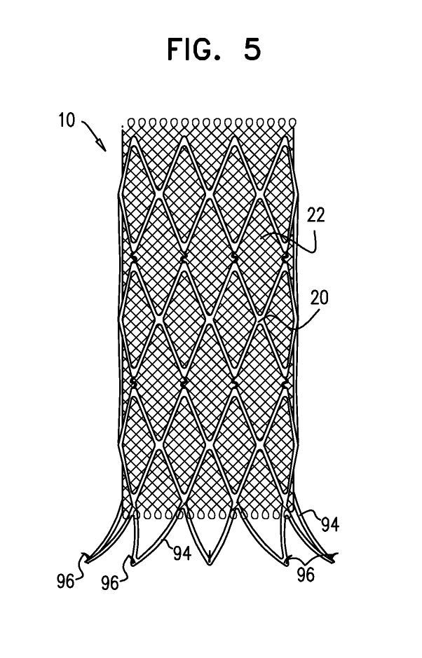

For some applications, proximal and/or distal portions of stent system 10 (typically of outer stent 20) comprise anchoring elements, for example as described hereinbelow with reference to FIG. 5, and/or in PCT Publication WO 2010/150208, mutatis mutandis, which is incorporated herein by reference, e.g., with reference to FIGS. 3, 7A-C, 9A-B, 10A-B, 13, 15A-C, 16, 17, 18, 19, 20A-B, and/or 21A-B thereof.

Reference is now made to FIGS. 4A-E, which are schematic illustrations of an exemplary method of deploying multi-component endovascular stent system 10 in the vicinity of an supra-renal abdominal aortic aneurysm 60 of an abdominal aorta 62, in accordance with an application of the present invention.

During a first stage of the implantation procedure, outer stent 20 is deployed in aorta 62 longitudinally spanning aortic aneurysm 60, i.e., from the distal to the proximal longitudinal ends of the aneurysm. In this exemplary deployment, a distal (upper) end of outer stent 20 is deployed slightly below aortic arch 82, and a proximal (lower) end of the outer stent is positioned above renal arteries 80. Although not shown in the figures, outer stent 20 is deployed using a delivery tool comprising a delivery catheter, such as described hereinbelow regarding the deployment of inner stent 22, with reference to FIGS. 4B-E. Outer stent 20 is transvascularly (typically percutaneously) introduced into aorta 62, e.g., via one of the iliac arteries, while the outer stent is positioned in the delivery catheter, restrained in its radially-compressed state by the catheter. The outer stent is deployed in the aorta by transitioning the outer stent to its radially-expanded state.

As shown in FIG. 4B, inner stent 22 is deployed using an endovascular stent delivery tool 70, which typically comprises a delivery catheter 72, a distal tip 74, and a guidewire 76. Inner stent 22 is initially positioned in delivery catheter 72, restrained in the stent's radially-compressed and longitudinally-elongated state by the catheter. Inner stent 22 is transvascularly (typically percutaneously) introduced into aorta 62, e.g., via one of the iliac arteries, while positioned in delivery catheter 72. The inner stent, while in catheter 72, is introduced at least partially into outer stent 20 (typically only a longitudinal portion of the inner stent is within the outer stent at this stage of the implantation procedure). In this exemplary deployment, delivery catheter 72 and distal tip 74 are advanced over guidewire 76 until the distal tip is positioned at or slightly below aortic arch 82.

Delivery catheter 72 is proximally withdrawn, releasing inner stent 22 in aorta 62, within outer stent 60, and transitioning the inner stent to a radially-expanded and longitudinally-contracted state thereof. FIG. 4C shows an early stage of deployment of the inner stent, while FIG. 4D shows the inner stent nearly fully deployed.

FIG. 4E shows inner stent 22 fully deployed, at least partially (e.g., entirely) nested within outer stent 20 such that inner and outer stents 22 and 20 are fixed together. As can be seen, inner stent 22 is nested within outer stent 20, and stent system 10 has been assembled in situ. Typically, when in its radially-expanded and longitudinally-contracted state, inner stent 22 has a contracted length that is no more than 25% of its elongated length prior to deployment, such as no more than 12.5%, no more than 10%, or no more than 8.3%.

Reference is again made to FIG. 3. For some applications, each of outer stent 20 and inner stent 22 further comprises one or more respective radiopaque markers 90A and 90B. For some applications, the radiopaque markers are disposed along either a proximal or distal end of inner stent 22 and/or outer stent 20. The radiopaque markers may help the surgeon properly longitudinally align the stents with the vascular lesion and relative to one another.

Reference is again made to FIGS. 4A-E. For some applications, outer stent 20 is shaped so as to define a plurality of inwardly-protruding fixation members 32, which are configured to facilitate coupling of inner stent 22 to outer stent 20. For some applications, at least one of (e.g., all of) inwardly-protruding fixation members 32 comprises a barb. For some applications, the plurality of inwardly-protruding fixation members 32 comprises at least a plurality of proximally-disposed fixation members 32, disposed near a proximal end of outer stent 20, and a plurality of distally-disposed fixation members 32, disposed near a distal end of the outer stent. Optionally, the proximally-disposed fixation members comprise respective distally- and inwardly-oriented barbs. Alternatively or additionally, the distally-disposed fixation members comprise respective proximally- and inwardly-oriented barbs.

For some applications, stent system 10 is shaped so as to define a side-facing fenestration, when inner stent 22 is within outer stent 20, when the inner stent is in its radially-expanded and longitudinally-contracted state within the outer stent, when the outer stent is in its radially-expanded state. For example, the side-facing fenestration may be generally circular. For some applications, a perimeter of the fenestration is between 10% and 50% of a perimeter of outer stent 20 adjacent the fenestration, when the outer stent is unconstrained in its radially-expanded state. This fenestrated configuration may be used in combination with any of the configurations described herein.

Reference is made to FIG. 5, which is a schematic illustration of another configuration of stent system 10, in accordance with an application of the present invention. In this configuration, outer stent 20 further comprises a plurality of outwardly protruding-fixation elements 94, which are configured to facilitate fixation of the outer stent to an inner wall of a body lumen. For example, fixation elements 94 may be positioned at a proximal end of outer stent 20, as shown; alternatively or additionally, fixation elements 94 may be positioned at a distal end of the outer stent (not shown). For some applications, fixation elements 94 comprise barbs 96. For some applications, fixation elements (e.g., barbs) are configured as shown in FIGS. 1, 2, 3, 5A-B, 7A, 7B, 7C, 9A-D, 10A, 10B, 13, 15A-C, 16, 17, 18, 19, 20, and/or 20A-B of the above-mentioned '208 publication, mutatis mutandis. This configuration may be used in combination with any of the configurations described herein.

Stent system 10 may be deployed alone, or as a component of a larger stent system comprising additional stents, for example as described with reference to FIGS. 4E and/or 21B of the '208 publication, mutatis mutandis, or in PCT Publication WO 08/107,885, mutatis mutandis, which is incorporated herein by reference. For some applications, stent system 10 defines a single lumen, while for other applications, the stent system 10 defines a plurality of lumen, e.g., is bifurcated, such as described with reference to FIG. 3 of the above-mentioned '208 publication, mutatis mutandis.

Although the endovascular stent system is generally described herein as being deployed via an iliac artery and the aorto-iliac bifurcation, for some applications, the prostheses are instead deployed via a subclavian artery. Furthermore, although the endovascular stent system is generally described herein as being deployed in the aorta, the system may also be deployed in another blood vessel, such as another artery, e.g., an aneurysmatic artery, such as an aneurysmatic iliac artery.

As used in the present application, including in the claims, “tubular” means having the form of an elongated hollow object that defines a conduit therethrough. A “tubular” structure may have varied cross-sections therealong, and the cross-sections are not necessarily circular. For example, one or more of the cross-sections may be generally circular, or generally elliptical but not circular, or circular.

The scope of the present invention includes embodiments described in the following applications, which are assigned to the assignee of the present application and are incorporated herein by reference. In an embodiment, techniques and apparatus described in one or more of the following applications are combined with techniques and apparatus described herein:
PCT Application PCT/IL2008/000287, filed Mar. 5, 2008, which published as PCT Publication WO 2008/107885 to Shalev et al., and U.S. application Ser. No. 12/529,936 in the national stage thereof, which published as US Patent Application Publication 2010/0063575 to Shalev et al.
U.S. Provisional Application 60/892,885, filed Mar. 5, 2007
U.S. Provisional Application 60/991,726, filed Dec. 2, 2007
U.S. Provisional Application 61/219,758, filed Jun. 23, 2009
U.S. Provisional Application 61/221,074, filed Jun. 28, 2009
PCT Application PCT/IB2010/052861, filed Jun. 23, 2010, which published as PCT Publication WO 2010/150208
PCT Application PCT/IL2010/000564, filed Jul. 14, 2010, which published as PCT Publication WO 2011/007354
PCT Application PCT/IL2010/000917, filed Nov. 4, 2010, which published as PCT Publication WO 2011/055364
PCT Application PCT/IL2010/000999, filed Nov. 30, 2010, which published as PCT Publication WO 2011/064782
PCT Application PCT/IL2010/001018, filed Dec. 2, 2010, which published as PCT Publication WO 2011/067764
PCT Application PCT/IL2010/001037, filed Dec. 8, 2010, which published as PCT Publication WO 2011/070576
PCT Application PCT/IL2011/000135, filed Feb. 8, 2011, which published as PCT Publication WO 2011/095979
U.S. application Ser. No. 13/031,871, filed Feb. 22, 2011, which published as US Patent Application Publication 2011/0208289
U.S. Provisional Application 61/496,613, filed Jun. 14, 2011
U.S. Provisional Application 61/505,132, filed Jul. 7, 2011

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. The apparatus according to claim 74,
wherein the inner stent (a) has (i) an elongated length when in its radially-compressed and longitudinally-elongated state and (ii) a contracted length when in its radially-expanded and longitudinally-contracted state within the outer stent when the outer stent is unconstrained in a radially-expanded state, and (b) is configured such that a ratio of the elongated length to the contracted length is at least 4.

2. The apparatus according to claim 1, wherein the ratio is at least 8.

3. (canceled)

4. The apparatus according to claim 2, wherein the ratio is at least 12.

5-7. (canceled)

8. The apparatus according to claim 74, wherein the inner stent further comprises a thrombogenic agent that coats the wire of the braided wire mesh.

9. (canceled)

10. The apparatus according to claim 74, wherein the multi-component endovascular stent system does not comprise any graft material attached to the inner stent at least when the inner stent is not nested in the outer stent.

11. The apparatus according to claim 1,
wherein the inner stent has an expanded diameter when in its radially-expanded and longitudinally-contracted state within the outer stent when the outer stent is unconstrained in its radially-expanded state,
wherein the inner stent has a contracted diameter when in its radially-compressed and longitudinally-elongated state, and
wherein a ratio of the expanded diameter to the contracted diameter is at least 1.1.

12. The apparatus according to claim 11, wherein the ratio of the expanded diameter to the contracted diameter is at least 1.5.

13. The apparatus according to claim 1, wherein the contracted length of the inner stent is equal to between 90% and 110% of a length of the outer stent when unconstrained in its radially-expanded state.

14. The apparatus according to claim 74, wherein a wall thickness of the inner stent when in its radially-expanded and longitudinally-contracted state within the outer stent, when unconstrained in its radially-expanded state, is no more than 200% of a wall thickness of the outer stent when unconstrained in a radially-expanded state.

15. The apparatus according to claim 74, wherein an expanded surface coverage ratio of the inner stent when in its radially-expanded and longitudinally-contracted state within the outer stent, when the outer stent is unconstrained in a radially-expanded state, is equal to at least 25% of a contracted surface coverage ratio of the inner stent when in its radially-compressed and longitudinally-elongated state.

16. The apparatus according to claim 15, wherein the expanded surface coverage ratio is equal to at least 50% of the contracted surface coverage ratio.

17. The apparatus according to claim 16, wherein the expanded surface coverage ratio is equal to at least 100% of the contracted surface coverage ratio.

18. The apparatus according to claim 74, wherein an expanded surface coverage ratio of the inner stent when in its radially-expanded and longitudinally-contracted state within the outer stent, when the outer stent is unconstrained in a radially-expanded state, is equal to at least 30% of an expanded surface coverage ratio of the outer stent when unconstrained in its radially-expanded state.

19. The apparatus according to claim 74, wherein an expanded surface coverage ratio of the inner stent when in its radially-expanded and longitudinally-contracted state within the outer stent, when the outer stent is unconstrained in a radially-expanded state, is equal to at least 20%.

20. The apparatus according to claim 19, wherein the expanded surface coverage ratio of the inner stent when in its radially-expanded and longitudinally-contracted state within the outer stent, when the outer stent is unconstrained in its radially-expanded state, is equal to at least 50%.

21. The apparatus according to claim 74,
wherein the braided wire mesh of the inner stent, if projected onto a plane when the inner stent is in its radially-expanded and longitudinally-contracted state within the outer stent, when the outer stent is unconstrained in a radially-expanded state, would define a plurality of openings, and
wherein, for each of at least 90% of the openings, every point on the plane within the projected opening is within 0.2 mm of a border of the opening on the plane, which border is defined by the braided wire mesh projected onto the plane.

22. The apparatus according to claim 74, wherein the braided wire mesh of the inner stent, if projected onto a plane when the inner stent is in its radially-expanded and longitudinally-contracted state within the outer stent, when the outer stent is unconstrained in a radially-expanded state, would define a plurality of openings, which have a median size of at least 100 square micrometers.

23. (canceled)

24. The apparatus according to claim 74, wherein the outer stent is shaped so as to define a plurality of outwardly-protruding fixation members, which are configured to facilitate fixation of the outer stent to an inner wall of a body lumen.

25. The apparatus according to claim 74, wherein the outer stent is shaped so as to define a plurality of inwardly-protruding fixation members, which are configured to facilitate coupling of the inner stent to the outer stent.

26-29. (canceled)

30. The apparatus according to claim 74, wherein the wire of the braided wire mesh has a diameter of between 50 and 200 micrometers.

31. (canceled)

32. The apparatus according to claim 74, wherein the outer stent comprises a plurality of structural stent elements, which are arranged as a plurality of circumferential bands.

33. (canceled)

34. The apparatus according to claim 32, wherein the outer stent further comprises a polymeric fabric, which connects the circumferential bands.

35. The apparatus according to claim 34, wherein the polymeric fabric covers less than an entire circumference of the outer stent along at least a portion of the outer stent.

36. (canceled)

37. The apparatus according to claim 35, wherein the polymeric fabric circumscribes a circumferential arc that has an arc angular center that varies along at least the portion, thereby providing the polymeric fabric with a generally helical shape.

38. A method for treating an aneurysm in an aneurismal body lumen of a human subject, the method comprising:
transvascularly introducing an outer generally tubular stent of a stent system into the aneurismal body lumen, while the outer stent is in a radially-compressed state thereof;
deploying the outer stent in the aneurismal body lumen longitudinally spanning the aneurysm by transitioning the outer stent to a radially-expanded state thereof, in which state the outer stent provides anchoring for the stent system within the aneurismal body lumen;
transvascularly introducing, into the aneurismal body lumen and at least partially into the outer stent, an inner generally tubular stent of the stent system, which inner tubular stent comprises a braided metal wire mesh, while the inner stent in a radially-compressed and longitudinally-elongated state thereof; and
deploying the inner stent in the aneurismal body lumen at least partially nested within the outer stent such that the inner and the outer stents are fixed together, by transitioning the inner stent to a radially-expanded and longitudinally-contracted state thereof, in which state the inner stent is substantially blood-impermeable and provides a generally blood-impervious fluid flow path through the stent system, beginning no later than four weeks after implantation of the outer and the inner stents in the aneurismal body lumen.

39. The method according to claim 70, wherein the contracted length is no more than 12.5% of the elongated length.

40. (canceled)

41. The method according to claim 39, wherein the contracted length is no more than 8.3% of the elongated length.

42-45. (canceled)

46. The method according to claim 38, wherein the inner stent further comprises a thrombogenic agent that coats the wire of the braided wire mesh.

47. (canceled)

48. The method according to claim 38, wherein transvascularly introducing the inner stent comprises transvascularly introducing the inner stent with no graft material attached to the inner stent.

49-58. (canceled)

59. The method according to claim 38,
wherein deploying the inner stent comprises transitioning the inner stent to its radially-expanded and longitudinally-contracted state within the outer stent when in its radially-expanded state, in which state the braided wire mesh of the inner stent, if projected onto a plane, would define a plurality of openings, and
wherein, for each of at least 90% of the openings, every point on the plane within the projected opening is within 0.2 mm of a border of the opening on the plane, which border is defined by the braided wire mesh projected onto the plane.

60. The method according to claim 38, wherein deploying the inner stent comprises transitioning the inner stent to its radially-expanded and longitudinally-contracted state within the outer stent when in its radially-expanded state, in which state the braided wire mesh of the inner stent, if projected onto a plane, would define a plurality of openings, which have a median size of at least 100 square micrometers.

61-69. (canceled)

70. The method according to claim 38, wherein transitioning the inner stent to the radially-expanded and longitudinally-contracted state comprises transitioning the inner stent to the radially-expanded and longitudinally-contracted state in which the inner stent has a contracted length that is no more than 25% of an elongated length of the inner state when in the radially-compressed and longitudinally-elongated state thereof.

71. The method according to claim 70, wherein (a) a ratio of the elongated length to the contracted length of the inner stent, divided by (b) a ratio of a length of the outer stent when in its radially-compressed state to a length of the outer stent when in its radially-expanded state, is at least 4.

72. The method according to claim 38, wherein the outer stent includes: (a) a plurality of structural stent elements, which are arranged as a plurality of circumferential bands, and (b) a polymeric fabric, which (i) connects the circumferential bands, (ii) covers less than an entire circumference of the outer stent along at least a portion of the outer stent, and (iii) circumscribes a circumferential arc that has an arc angular center that varies along at least the portion, thereby providing the polymeric fabric with a generally helical shape.

73. The method according to claim 38,
wherein transitioning the outer stent to the radially-expanded state comprises transitioning the outer stent to the radially-expanded state in which outer stent defines a first plurality of openings,
wherein transitioning the inner stent to the radially-expanded and longitudinally-contracted state comprises transitioning the inner stent to the radially-expanded and longitudinally-contracted state in which the inner stent defines a second plurality of openings, and
wherein and each of the openings of the outer stent overlies more than one of the openings of the inner stent.

74. Apparatus comprising a multi-component endovascular stent system, which comprises:
an outer generally tubular stent and an inner generally tubular stent, the outer and inner stents being configured to be assembled in situ by nesting the inner stent in the outer stent, with the outer stent being configured to provide anchoring for the stent system within an aneurismal body lumen and with the inner stent being configured to provide a generally blood-impervious fluid flow path through the stent system,
wherein the inner stent comprises a braided metal wire mesh configured to assume (a) a radially-compressed and longitudinally-elongated state for low-crossing-profile endoluminal delivery, and (b) a radially-expanded and longitudinally-contracted state for secure fixation within the outer stent, with the inner stent itself being configured to be substantially blood-impervious when in its radially-expanded and longitudinally-contracted state, beginning no more than four weeks after implantation of the stent system in a body lumen.

75. The apparatus according to claim 74, wherein, when the outer stent is in a radially-expanded state and the inner stent is in its radially-expanded and longitudinally-contracted state securely fixed within the outer stent, the inner and the outer stents define respective pluralities of openings, and each of the openings of the outer stent overlies more than one of the openings of the inner stent.

76. The apparatus according to claim 1, wherein (a) a ratio of the elongated length to the contracted length of the inner stent, divided by (b) a ratio of a length of the outer stent when in a radially-compressed state to a length of the outer stent when in its radially-expanded state, is at least 4.
